(19) United States
(12) Patent Application Publication
(10) Pub. No.: US 2011/0097365 A1
(43) Pub. Date: Apr. 28, 2011

(54) S-TRIAZINE DERIVATIVES CONTAINING AT LEAST TWO PARTICULAR SILANE AMINOBENZOATE OR SILANE AMINOBENZAMIDE GROUPS; PHOTOPROTECTIVE COSMETIC COMPOSITIONS CONTAINING THESE DERIVATIVES; USES OF THE SAID S-TRIAZINE DERIVATIVES

(75) Inventor: Herve Richard, Les Pavillons Sous Bois (FR)

(73) Assignee: L'OREAL, Paris (FR)

(21) Appl. No.: 12/739,638

(22) PCT Filed: Sep. 9, 2008
(86) PCT No.: PCT/EP2008/061927

§ 371 Date: Jan. 14, 2011

Related U.S. Application Data
(60) Provisional application No. 60/996,207, filed on Nov. 6, 2007.

(30) Foreign Application Priority Data
Oct. 25, 2007 (FR) 07/58578

(51) Int. Cl. A61K 8/02; C07D 251/48; C07D 251/54; A61K 8/49; A61K 8/27; A61Q 17/04

(52) U.S. Cl. 424/401; 544/208; 544/197; 424/59

(57) ABSTRACT

The invention relates to novel s-triazine derivatives containing at least two particular silane aminobenzoate or silane aminobenzamide groups and to their cosmetic uses. The invention also relates to photoprotective compositions comprising s-triazine derivatives containing at least two particular grafted silane aminobenzoate or silane aminobenzamide groups as sunscreens that are active in the UV-B radiation range.

The invention relates to novel s-triazine derivatives containing at least two particular silane aminobenzoate or silane aminobenzamide groups and to their cosmetic uses.

The invention also relates to photoprotective compositions comprising s-triazine derivatives containing at least two grafted silane aminobenzoate or silane aminobenzamide groups as sunscreens that are active in the UV radiation range.

It is well known that light radiation with wavelengths of between 280 nm and 400 nm permit tanning of the human epidermis and that rays with wavelengths of between 280 and 320 nm, which are known as UV-B rays, cause skin burns and erythema that can harm the development of a natural tan; this UV-B radiation should thus be screened out.

It is also known that UV-A rays, with wavelengths of between 320 and 400 nm, which cause tanning of the skin, are liable to induce an impairment in the skin, especially in the case of sensitive skin or skin that is continually exposed to solar radiation. UV-A rays in particular bring about a loss of elasticity of the skin and the appearance of wrinkles, leading to premature ageing. They promote the triggering of the erythemal reaction or amplify this reaction in certain individuals and may even be the cause of phototoxic or photoallergic reactions. It is thus desirable also to screen out UV-A radiation.

UV-A and UV-B rays should thus be screened out, and cosmetic compositions for protecting the human epidermis containing UV-A and UV-B-screening agents currently exist.

These antisun compositions are quite often in the form of an emulsion, of oil-in-water type (i.e. a cosmetically and/or dermatologically acceptable support consisting of an aqueous dispersing continuous phase and of a fatty dispersed discontinuous phase), or of water-in-oil type (aqueous phase dispersed in a continuous fatty phase), which contains, in varying concentrations, one or more standard liposoluble organic screening agents and/or standard water-soluble organic screening agents capable of selectively absorbing the harmful UV radiation, these screening agents (and the amounts thereof) being selected as a function of the desired sun protection factor, the sun protection factor (SPF) being expressed mathematically as the ratio of the dose of UV radiation required to reach the erythema-forming threshold with the UV-screening agent to the dose of the UV radiation required to reach the erythema-forming threshold without the UV-screening agent. In such emulsions, the hydrophilic screening agents are present in the aqueous phase and the lipophilic screening agents are present in the fatty phase.

Besides their screening power, the desired photo-protective compounds should also have good cosmetic properties, good solubility or good dispersibility in the usual solvents and especially in fatty substances such as oils and fats, and also good resistance to water and perspiration (remanence) and satisfactory photostability.

The most commonly used UV-screening agents are organic and soluble in oils or in aqueous media; they generally have in their structure a chromophoric group attached to a solubilizing group, which is generally a fatty chain in the case of liposoluble UV-screening agents or a carboxylic or sulfonic acid group in the case of water-soluble UV-screening agents.

Insoluble, and especially micronized, organic UV-screening agents, with a mean particle size ranging from 10 nm to 2 μm, which have the advantage of being more effective than their soluble homologues comprising the same chromophoric group in an equivalent amount, are known in the prior art. Micronized insoluble UV-screening agents of this type are especially described in patents GB-A-2 303 549 and EP-A-893 119. They are generally obtained in the form of an aqueous dispersion of particles via a process of milling an insoluble organic UV-screening agent in the form of coarse particles in the presence of a suitable surfactant for improving the dispersion of the particles thus obtained in the cosmetic formulations. An example of a process for micronizing insoluble organic screening agents is described in patent applications GB-A-2 303 549 and EP-A-893 119. The milling machine used according to these documents may be a jet mill, a ball mill, a vibration mill or a hammer mill, and preferably a high spin-speed mill or an impact mill and more particularly a rotary ball mill, a vibrating mill, a tube mill or a rod mill.

It is also known in patent application WO 99/66896 that by incorporating these micronized insoluble UV-screening agents in an oily phase of an antisun formulation such as emulsions, the sun protection is improved. To allow their incorporation into the oily phase of these emulsions, it is necessary to treat the aqueous dispersions of micronized UV-screening agents as described previously either by converting them into powder, for example by precipitation in a solvent or by atomization; the said particles possibly being coated with a surfactant (phospholipids) or a polymer (polyvinylpyrrolidone, polyacrylate, etc.) so as to avoid aggregation phenomena. Another technique consists in performing a phase transfer on the aqueous dispersion of micronized UV-screening agent, by placing the dispersion in contact with the oily phase, followed by evaporating off the water under vacuum according to standard techniques. According to patent application WO 2006/024 633, a micronized insoluble UV-screening agent may also be incorporated into the continuous oily phase of a water-in-oil emulsion by combining it with a C₈-C₂₈ fatty alcohol. The industrial manufacture of micronized insoluble UV-screening agents suspended in an oily phase is thus complex and expensive on account of these various necessary steps.

There is still a need to find novel insoluble UV-screening agents that may be effective in absorbing UV radiation and that are readily dispersible in cosmetic oils without it being necessary to resort to powder-forming or phase-transfer techniques starting with an aqueous dispersion of micronized UV-screening agent, or to resort to the addition of a fatty alcohol.

The Applicant has discovered, surprisingly, a novel family of insoluble UV-screening agents that have good absorbing properties in the range of UV-B rays and that show spontaneous dispersibility in cosmetic oils. These are novel s-triazine derivatives bearing at least two silane aminobenzoate or silane aminobenzamide groups and corresponding to formula (I) which will be detailed hereinbelow. These derivatives also show good photostability and also good cosmetic properties.

The invention thus relates to a novel family of s-triazine derivatives bearing at least two particular silane aminobenzoate or silane aminobenzamide groups of formula (I) which will be defined in detail hereinbelow.

The invention also relates to a cosmetic or dermatological composition for photoprotecting keratin materials, containing, in a cosmetically acceptable medium, at least one compound of formula (I).

The invention also relates to the cosmetic use of a compound of formula (I), as an agent for screening out UV-B radiation.

The term “cosmetically acceptable” refers to an agent that is compatible with the skin and/or its integuments, which has a pleasant colour, odour and feel, and which does not cause any unacceptable discomfort (stinging, tautness or redness) liable to put the consumer off using this composition.

Other subjects will become apparent in the light of the description.

The compounds in accordance with the present invention correspond to the general formula (I) below:

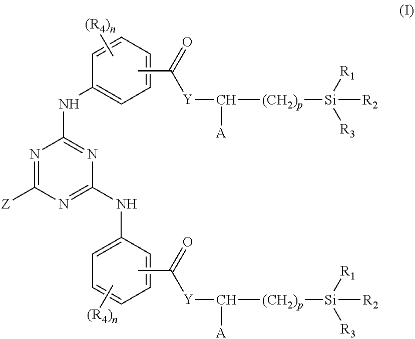

in which:
the radicals R₁, R₂ and R₃, which may be identical or different, represent a linear or branched, saturated or unsaturated C₁-C₁₂ alkyl radical, which may comprise one or more halogen atoms (for example Cl, Br or F); a phenyl radical,
p is equal to 0 or 1,
Y denotes —O— or —NR₅,
the radical R₅ denotes hydrogen, a linear or branched C₁-C₈ alkyl radical or a C₆-C₁₂ aryl radical,
A denotes hydrogen, a linear or branched, saturated or unsaturated C₁-C₈ alkyl radical, a phenyl radical or a group Si(CH₃)₃, with the proviso that when A is Si(CH₃)₃, then p=0 and R₁, R₂ and R₃ are methyl,
the radical R₄ denotes a linear or branched, saturated or unsaturated C₁-C₂₀ alkyl radical, an OH radical, a linear or branched C₁-C₂₀ alkoxy radical or a group —(C═O)—Y—R₆,
n is equal to 0, 1 or 2,
the radical R₆ denotes hydrogen or a linear or branched C₁-C₂₀ alkyl radical,
the group (CO)—Y—CHA-(CH₂)ₚ—SiR₁R₂R₃ may be in the meta or para position relative to the amino group,
Z represents:
   (i) a linear or branched C₁-C₂₀ alkyl group,
   (ii) a linear or branched C₂-C₂₀ hydroxyalkyl group,
   (iii) a linear or branched C₁-C₂₀ alkoxy group,
   (iv) a para-alkoxyphenyl group of the type:

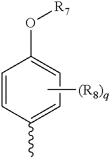

in which R₇ represents
hydrogen;
a linear or branched C₁-C₂₀ alkyl group;
a C₆-C₁₀ aryl group;
a group —(CH₂CHR₉—O)ₘR₁₀ or a group —CH₂—CH(OH)—CH₂—O—R₁₀ in which:
   R₉ represents hydrogen or methyl,
   R₁₀ represents hydrogen or a linear or branched C₁-C₈ alkyl group,
   m=1-20,
the radicals R₈, which may be identical or different, represent a linear or branched C₁-C₈ alkyl group, an OH radical or a linear or branched C₁-C₈ alkoxy group, this alkoxy group possibly forming with the ortho group OR₇ a C₁-C₂ dioxyalkylene ring, with the following exclusion that R₈ cannot be a hydroxyl radical in the meta position relative to the group OR₇,
q=0-3,
   (v) the radical NHR₁₁ or the radical N(R₁₁)₂ with R₁₁ being a linear or branched C₁-C₂₀ alkyl radical,
   (vi) the group:

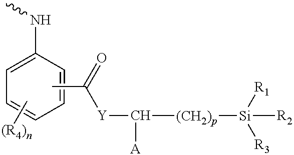

(vii) or the radical —NH—W in which W represents a chromophore for screening out UV radiation, chosen in particular from the group constituted by a benzylidenecamphor, a benzalmalonate, a benzoate, a salicylate, an anthranilate, a benzotriazole, a benzimidazole, a benzoxazole, a benzothiazole, a cinnamate or a cinnamonitrile.

In formula (I) above, the alkyl radicals may be linear or branched, saturated or unsaturated, and chosen especially from methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The alkyl radical that is particularly preferred is the methyl radical.

In formula (I) above, the aryl radicals are preferably phenyl.

The S-triazine derivatives bearing at least two particular silane aminobenzoate groups of formula (I) preferably have at least one, and even more preferentially all, of the following characteristics:
R₁ to R₃ denote a C₁-C₄ alkyl and more preferentially methyl,
p is 0,
A is H or Si(CH₃)₃,
Y is —O— or —NH—,
n is 0 or 1,
Z is a silane aminobenzoate or para-methoxyphenyl group,
F is a benzoate or benzylidenecamphor group.

Among the compounds of formula (I) that are more particularly preferred, mention will be made of those chosen from the compounds of formulae (a) to (g) below:

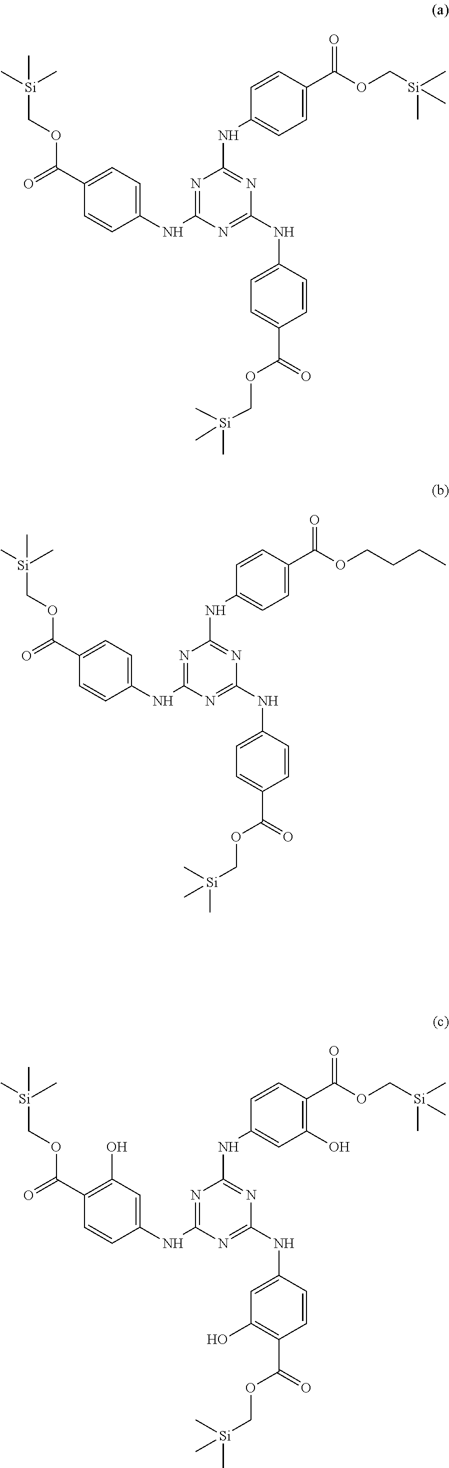

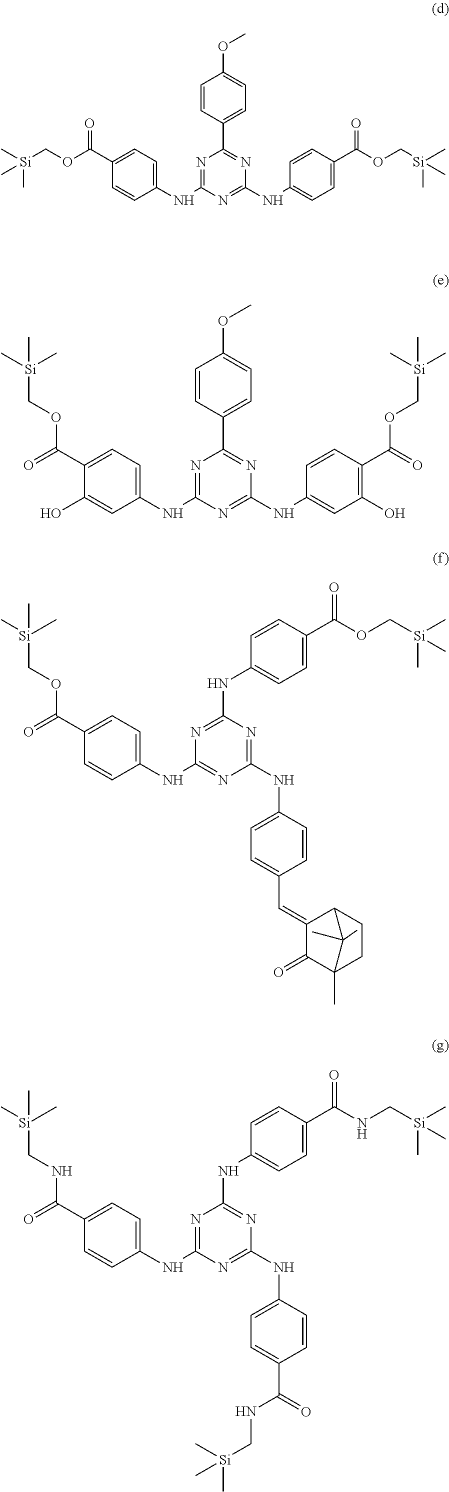

According to one particular mode of the invention, the insoluble compounds of formula (I) are in micronized form and their mean particle size is between 10 nm and μm.

The particles preferably have a mean size of between 10 nm and 2 μm and more preferentially between 20 nm and 2 μm.

The mean size of the particles may be determined by any standard method, such as optical methods (quasi-elastic light scattering or laser scattering), centrifugation methods or microscopic visualization methods and image analysis.

The insoluble organic screening agents according to the invention may be brought into the particle form having the desired mean size by any suitable means, such as dry milling or milling in a solvent medium, screening, atomization, micronization or spraying.

The derivatives of formula (I) may be obtained according to Scheme A below:

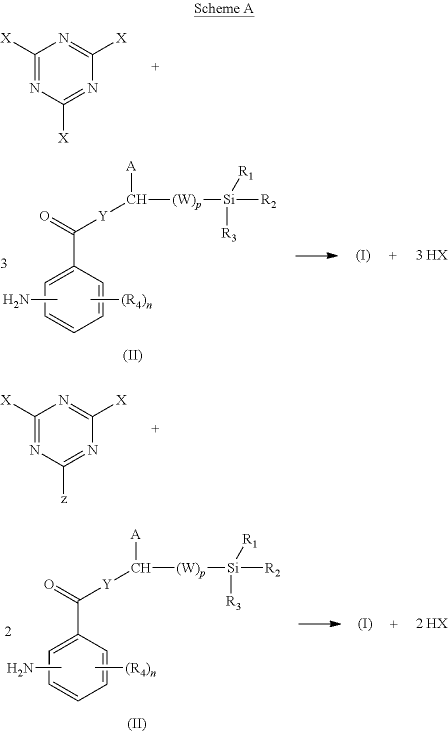

in which R₁, R₂, R₃, R₄, A, Z, Y, n and p have the definition of formula (I) above and X represents a halogen, in particular chlorine or bromine.

The above reactions may be optionally performed without solvent or in the presence of a solvent (for example: toluene, xylene or acetone/water), at a temperature of between 0° C. and 250° C. and more particularly between 5° C. and 150° C.

The compounds of formula (II) may be prepared according to known methods described, for example, in the Bulletin of the Academy of Sciences of the USSR, Division of Chemical Sciences 4, pages 841-2 (1984) according to Scheme B below:

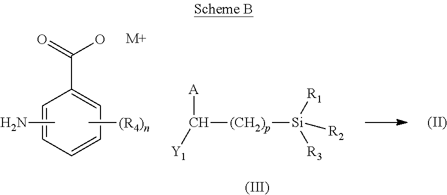

in which R₁, R₂, R₃, R₄, A, n and p have the definition of formula (I) above, Y₁ represents a halogen, in particular chlorine, bromine or iodine, and M+ represents an alkali metal such as sodium or potassium.

The compounds of formula (I) may also be obtained by transesterification of derivatives of formula (III) according to Scheme C below:

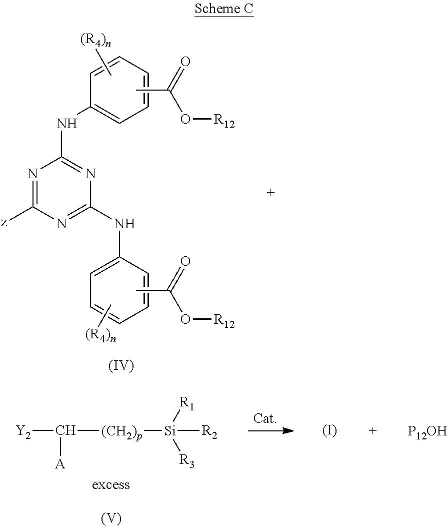

in which R₁, R₂, R₃, R₄, A, Z, n and p have the definition of formula (I) above, R₁₂ is methyl or ethyl and Y₂ represents OH or NH₂.

As silane halide derivatives of formula (III), mention may be made of the following commercial products: chloromethyltrimethylsilane (RN 2344-80-1) sold by the company Wacker, iodomethyltrimethylsilane (RN 42 06-67-1), (chloromethyl)dimethylethylsilane (RN 3121-77-5), (chloromethyl)dimethyl-n-butylsilane (RN 3121-75-3), (chloromethyl)dimethylpentylsilane (RN 73013-39-5), (chloromethyl)dodecyldimethylsilane (RN 70851-47-7), (chloromethyl)triethylsilane (RN 757-34-6), 2-chloro-ethyltrimethylsilane (RN 7787-87-3), bis(trimethyl-silyl)methyl chloride (RN 5926-35-2), (chloromethyl)-dimethylphenylsilane (RN 1833-51-8), (chloromethyl)-diphenylmethylsilane (RN 18407-40-4) and (trimethyl-silylmethyl)dimethylchloromethylsilane (RN 18306-73-5).

As derivatives of formula (V) in which Y₂ is OH, mention may be made of hydroxymethyltrimethylsilane (RN 3219-63-4) or 2-(trimethylsilyl)ethanol (RN 2916-68-9), which are commercial products.

As derivatives of formula (V) in which Y₂ is NH₂, mention may be made of aminomethyltrimethylsilane (RN 18166-02-4) sold by the company Gelest, and also (phenyl)(trimethylsilyl)methylamine or bis(trimethyl-silyl)methylamine (RN 134340-00-4).

The solubility in oils of the dispersible screening agents of the invention may be defined as being less than 3% and more particularly less than 1% at 25° C.

The cosmetic oils used may especially be liquid petroleum jelly, a mixture of fatty acid triglycerides, for instance caprylic/capric triglyceride (Miglyol 812), phenethyl benzoate (X-Tend), isopropyl lauroyl sarcosinate (Eldew SL), alkyl benzoates (Finsolv TN) or ethylhexyl methoxycinnamate (Parsol MCX).

These screening agents are also water-insoluble (solubility of less than 1% at 25° C.).

The compounds of formula (I) are generally present in the composition of the invention in proportions of between 0.01% and 20% by weight and preferably between 0.1% and 10% by weight relative to the total weight of the composition.

The compositions in accordance with the invention may also comprise other additional UV-A-active and/or UV-B-active organic or mineral UV-screening agents, which are water-soluble or liposoluble, or even insoluble in the commonly used cosmetic solvents.

The additional organic UV-screening agents are chosen especially from anthranilates; cinnamic derivatives; dibenzoylmethane derivatives; salicylic derivatives; camphor derivatives; benzophenone derivatives; β,β-diphenylacrylate derivatives; triazine derivatives other than those of formula (I); benzotriazole derivatives; benzalmalonate derivatives, especially those mentioned in patent U.S. Pat. No. 5,624,663; benzimidazole derivatives; imidazolines; bis-benzazolyl derivatives as described in Patents EP 669 323 and U.S. Pat. No. 2,463,264; p-aminobenzoic acid (PABA) derivatives; methylenebis-(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; α-alkylstyrene-based dimers, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981 and mixtures thereof.

As examples of additional organic UV-screening agents, mention may be made of those denoted hereinbelow under their INCI name:

### Para-Aminobenzoic Acid Derivatives:

PABA,
Ethyl PABA,
Ethyl dihydroxypropyl PABA,
Ethylhexyl dimethyl PABA sold especially under the name
Escalol 507 by ISP,
Glyceryl PABA,
PEG-25 PABA sold under the name Uvinul P25 by BASF.

### Dibenzoylmethane Derivatives:

Butylmethoxydibenzoylmethane sold especially under the trade name Parsol 1789 by DSM,
Isopropyldibenzoylmethane sold especially under the trade name Eusolex 8020 by Merck.

### Salicylic Derivatives:

Homosalate sold under the name Eusolex HMS by Rona/EM Industries,
Ethylhexyl salicylate sold under the name Neo Heliopan OS by Haarmann and Reimer,
Dipropylene glycol salicylate sold under the name Dipsal by Scher,
TEA salicylate sold under the name Neo Heliopan TS by Haarmann and Reimer.

### Cinnamic Derivatives:

Ethylhexyl methoxycinnamate sold in particular under the trade name Parsol MCX by DSM,
Isopropyl methoxycinnamate,
Isoamyl methoxycinnamate sold under the trade name Neo Heliopan E 1000 by Haarmann and Reimer,
Cinoxate,
DEA methoxycinnamate,
   Diisopropyl methylcinnamate,
Glyceryl ethylhexanoate dimethoxycinnamate.

### β,β-Diphenylacrylate Derivatives:

Octocrylene sold in particular under the trade name Uvinul N539 by BASF,
Etocrylene sold in particular under the trade name Uvinul N35 by BASF.

### Benzophenone Derivatives:

Benzophenone-1 sold under the trade name Uvinul 400 by BASF,
Benzophenone-2 sold under the trade name Uvinul D50 by BASF,
Benzophenone-3 or Oxybenzone sold under the trade name Uvinul M40 by BASF,
Benzophenone-4 sold under the trade name Uvinul MS40 by BASF,
Benzophenone-5,
Benzophenone-6 sold under the trade name Helisorb 11 by Norquay,
Benzophenone-8 sold under the trade name Spectra-Sorb UV-24 by American Cyanamid,
Benzophenone-9 sold under the trade name Uvinul DS-49 by BASF,
Benzophenone-12
n-hexyl 2-(4-diethylamino-2-hydroxybenzoyl)benzoate sold under the trade name Uvinul A+ or in the form of a mixture with ethylhexyl methoxycinnamate such as the product sold under the trade name Uvinul A+B by the company BASF.

### Benzylidenecamphor Derivatives:

3-Benzylidenecamphor manufactured under the name Mexoryl SD by Chimex,
4-Methylbenzylidenecamphor sold under the name Eusolex 6300 by Merck,
Benzylidenecamphorsulfonic acid manufactured under the name Mexoryl SL by Chimex,
Camphor benzalkonium methosulfate manufactured under the name Mexoryl SO by Chimex,
Terephthalylidenedicamphorsulfonic acid manufactured under the name Mexoryl SX by Chimex,
Polyacrylamidomethylbenzylidenecamphor manufactured under the name Mexoryl SW by Chimex.

### Phenylbenzimidazole Derivatives:

Phenylbenzimidazolesulfonic acid sold in particular under the trade name Eusolex 232 by Merck,
Disodium phenyldibenzimidazoletetrasulfonate sold under the trade name Neo Heliopan AP by Haarmann and Reimer.

### Phenylbenzotriazole Derivatives:

Drometrizole trisiloxane sold under the name Silatrizole by Rhodia Chimie,
Methylenebis(benzotriazolyl)tetramethylbutylphenol sold in solid form under the trade name MIXXIM BB/100 by Fairmount Chemical, or in micronized form as an aqueous dispersion under the trade name Tinosorb M by Ciba Specialty Chemicals.

### Triazine Derivatives:

bis-Ethylhexyloxyphenol methoxyphenyl triazine sold under the trade name Tinosorb S by Ciba Geigy,
Ethylhexyl triazone sold in particular under the trade name Uvinul T150 by BASF,
Diethylhexyl butamido triazone sold under the trade name Uvasorb HEB by Sigma 3V,
2,4,6-tris(Dineopentyl 4′-aminobenzalmalonate)-s-triazine, 2,4,6-tris(Diisobutyl 4′-aminobenzalmalonate)-s-triazine, the symmetrical triazine screening agents described in patent U.S. Pat. No. 6,225,467, patent application WO 2004/085 412 (see compounds 6 and 9) or the document “Symmetrical Triazine Derivatives” IP.COM Journal, IP.COM INC WEST HENRIETTA, N.Y., US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which is also mentioned in patent applications WO 06/035 000, WO 06/034 982, WO 06/034 991, WO 06/035 007, WO 2006/034 992 and WO 2006/034 985.

### Anthranilic Derivatives:

Menthyl anthranilate sold under the trade name Neo Heliopan MA by Haarmann and Reimer.

### Imidazoline Derivatives:

Ethylhexyldimethoxybenzylidenedioxoimidazoline propionate.

### Benzalmalonate Derivatives:

Dineopentyl 4′-methoxybenzalmalonate,
Polyorganosiloxane containing benzalmalonate functions, for instance Polysilicone-15, sold under the trade name Parsol SLX by Hoffmann LaRoche

### 4,4-Diarylbutadiene Derivatives:

1,1-Dicarboxy(2,2′-dimethylpropyl)-4,4-diphenylbuta-diene,

### Benzoxazole Derivatives:

2,4-bis[5-(dimethylpropyl)benzoxazol-2-yl(4-phenyl)-imino]-6-(2-ethylhexyl)imino-1,3,5-triazine sold under the name Uvasorb K2A by Sigma 3V
   and mixtures thereof.

The preferential additional organic UV-screening agents are chosen from:
Butylmethoxydibenzoylmethane,
Ethylhexyl methoxycinnamate,
Homosalate,
Ethylhexyl salicylate,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyldibenzimidazoletetrasulfonate,
Methylenebis(benzotriazolyl)tetramethylbutylphenol,
Ethylhexyl triazone,
bis-Ethylhexyloxyphenol methoxyphenyl triazine,
Diethylhexyl butamido triazone,
2,4,6-tris(Dineopentyl 4′-aminobenzalmalonate)-s-triazine,
2,4,6-tris(Diisobutyl 4′-aminobenzalmalonate)-s-triazine,
2,4,6-tris(Biphenyl-4-yl-1,3,5-triazine),
2,4,6-tris(Terphenyl)-1,3,5-triazine
Drometrizole trisiloxane,
Polysilicone-15,
Dineopentyl 4′-methoxybenzalmalonate,
1,1-Dicarboxy(2,2′-dimethylpropyl)-4,4-diphenylbutadiene,
2,4-Bis[5-(dimethylpropyl)benzoxazol-2-yl(4-phenyl)-imino]-6-(2-ethylhexyl)imino-1,3,5-triazine,
   and mixtures thereof.

The mineral UV-screening agents are chosen from coated or uncoated metal oxide pigments (mean size of the primary particles: generally between 5 nm and 100 nm and preferably between 10 nm and 50 nm), for instance titanium oxide (amorphous or crystallized in rutile and/or anatase form), iron oxide, zinc oxide, zirconium oxide or cerium oxide pigments, which are all UV-photoprotective agents that are well known per se.

The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal alkoxides (of titanium or of aluminium), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to the said silicon atoms.

The term “silicones” also includes the silanes required for their preparation, in particular alkyl silanes.

The silicones used for coating the nanopigments that are suitable for the present invention are preferably chosen from the group containing alkyl silanes, polydialkylsiloxanes and polyalkylhydrogenosiloxanes. Even more preferentially, the silicones are chosen from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminium compounds or silicon compounds, or mixtures thereof.

The coated pigments are more particularly titanium oxides that have been coated:
with silica, such as the product Sunveil from the company Ikeda and the product Eusolex T-AVO from the company Merck,
with silica and iron oxide, such as the product Sunveil F from the company Ikeda,
with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia,
with alumina, such as the products Tipaque TTO-55 (B) and Tipaque TTO-55 (A) from the company Ishihara and UVT 14/4 from the company Kemira,
with alumina and aluminium stearate, such as the product Microtitanium Dioxide MT 100 TV, MT 100 TX, MT 100 Z and MT-01 from the company Tayca, and the products Solaveil CT-10 W, Solaveil CT 100 and Solaveil CT 200 from the company Uniqema,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira, or the product SMT-100 WRS from the company Tayca,
with alumina and treated with a silicone, such as the products Tipaque TTO-55 (S) from the company Ishihara or UV Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is between 25 and 40 nm, such as the product sold under the trade name T 805 by the company Degussa Silices, TiO₂ treated with a polydimethylsiloxane and for which the mean size of the elementary particles is 21 nm, such as the product sold under the trade name 70250 Cardre UF TiO2SI3 by the company Cardre, anatase/rutile TiO₂ treated with a polydimethylhydrogenosiloxane and for which the mean size of the elementary particles is 25 nm, such as the product sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Myoshi Kasei under the name UFTR, by the company Tomen under the name ITS and by the company Tioxide under the name Tioveil AQ.

The uncoated zinc oxide pigments are, for example:
those sold under the name Z-Cote by the company Sunsmart;
those sold under the name Nanox by the company Elementis;
those sold under the name Nanogard WCD 2025 by the company Nanophase Technologies.

The coated zinc oxide pigments are, for example:
those sold under the name Z-Cote HP1 by the company Sunsmart (dimethicone-coated ZnO);
those sold under the name Zinc Oxide CS-5 by the company Toshibi (ZnO coated with polymethylhydrogeno-siloxane);
those sold under the name Nanogard Zinc Oxide FN by the company Nanophase Technologies (as a 40% dispersion in Finsolv TN, C₁₂-C₁₅ alkyl benzoate);
those sold under the name Daitopersion ZN-30 and Daitopersion ZN-50 by the company Daito (dispersions in cyclopolymethylsiloxane/oxyethylenated polydimethyl-siloxane, containing 30% or 50% of nanozinc oxides coated with silica and polymethylhydrogenosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol 2100 by the company ISP (alumina-treated ZnO dispersed in an ethylhexyl methoxycinnamate/PVP-hexadecene/methicone copolymer mixture);
those sold under the name Fuji ZnO-SMS-10 by the company Fuji Pigment (ZnO coated with silica and polymethylsilsesquioxane);
those sold under the name Nanox Gel TN by the company Elementis (ZnO dispersed at a concentration of 55% in C₁₂-C₁₅ alkyl benzoate with hydroxystearic acid polycondensate).

The uncoated cerium oxide pigments are sold under the name Colloidal Cerium Oxide by the company Rhone-Poulenc.

The uncoated iron oxide nanopigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220,

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, especially of titanium dioxide and of cerium dioxide, including the silica-coated equal-weight mixture of titanium dioxide and of cerium dioxide, sold by the company Ikeda under the name Sunveil A, and also the alumina, silica and silicone-coated mixture of titanium dioxide and of zinc dioxide, such as the product M 261 sold by the company Kemira, or the alumina, silica and glycerol-coated mixture of titanium dioxide and of zinc dioxide, such as the product M 211 sold by the company Kemira.

The additional UV-screening agents are generally present in the compositions according to the invention in proportions ranging from 0.01% to 20% by weight relative to the total weight of the composition, and preferably ranging from 0.1% to 10% by weight relative to the total weight of the composition.

The compositions according to the invention may also contain agents for artificially tanning and/or browning the skin (self-tanning agents) and more particularly dihydroxyacetone (DHA). They are preferably present in amounts ranging from 0.1% to 10% by weight relative to the total weight of the composition.

The compositions in accordance with the present invention may also comprise standard cosmetic adjuvants chosen especially from fatty substances, organic solvents, ionic or nonionic, hydrophilic or lipophilic thickeners, softeners, humectants, opacifiers, stabilizers, emollients, silicones, antifoams, fragrances, preserving agents, anionic, cationic, nonionic, zwitterionic or amphoteric surfactants, active agents, fillers, polymers, propellants, acidifying or basifying agents or any other ingredient usually used in cosmetics and/or dermatology.

According to one particular form of the invention, the compositions comprise at least one fatty phase.

The term “fatty phase” means any phase comprising one or more fatty substances.

The fatty substances may be constituted by an oil or a wax other than the apolar waxes as defined above, or mixtures thereof. The term “oil” means a compound that is liquid at room temperature. The term “wax” means a compound that is solid or substantially solid at room temperature and whose melting point is generally greater than 35° C.

Oils that may be mentioned include mineral oils (paraffin); plant oils (sweet almond oil, macadamia oil, grapeseed oil or jojoba oil); synthetic oils, for instance perhydrosqualene, fatty alcohols or fatty amides (for instance isopropyl lauroyl sarcosinate sold under the name Eldew SL-205 by the company Ajinomoto), fatty acids or fatty esters (for instance the C₁₂-C₁₅ alkyl benzoate sold under the trade name Finsolv TN or Witconol TN by the company Witco, octyl palmitate, isopropyl lanolate and triglycerides, including capric/caprylic acid triglycerides, and dicaprylyl carbonate sold under the name Cetiol CC by the company Cognis), oxyethylenated or oxypropylenated fatty esters and ethers; silicone oils (cyclomethicone and polydimethylsiloxanes, or PDMS) or fluoro oils, and polyalkylenes.

Waxy compounds that may be mentioned include carnauba wax, beeswax, hydrogenated castor oil, polyethylene waxes and polymethylene waxes, for instance the product sold under the name Cirebelle 303 by the company Sasol.

Among the organic solvents that may be mentioned are lower alcohols and polyols. These polyols may be chosen from glycols and glycol ethers, for instance ethylene glycol, propylene glycol, butylene glycol, dipropylene glycol or diethylene glycol.

Hydrophilic thickeners that may be mentioned include carboxyvinyl polymers such as the Carbopol products (carbomers) and the Pemulen products (acrylate/C10-C30-alkylacrylate copolymer); polyacrylamides, for instance the crosslinked copolymers sold under the names Sepigel 305 (CTFA name: polyacrylamide/C13-14 isoparaffin/Laureth 7) or Simulgel 600 (CTFA name: acryl-amide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name Hostacerin AMPS (CTFA name: ammonium polyacryloyldimethyltaurate) or Simulgel 800 sold by the company SEPPIC (CTFA name: sodium polyacryloyldimethyltaurate/polysorbate 80/sorbitan oleate); copolymers of 2-acrylamido-2-methylpropane-sulfonic acid and of hydroxyethyl acrylate, for instance Simulgel NS and Sepinov EMT 10 sold by the company SEPPIC; cellulose-based derivatives such as hydroxyethylcellulose; polysaccharides and especially gums such as xanthan gum; and mixtures thereof.

Lipophilic thickeners that may be mentioned include synthetic polymers such as poly(C₁₀-C₃₀ alkyl acrylates) sold under the name Intelimer IPA 13-1 and Intelimer IPA 13-6 by the company Landec, or modified clays such as hectorite and its derivatives, for instance the products sold under the name Bentone.

Among the active agents that may be mentioned are:
vitamins (A, C, E, K, PP, etc.) and derivatives or precursors thereof, alone or as mixtures;
antipollution agents and/or free-radical scavengers;
depigmenting agents and/or propigmenting agents;
antiglycation agents;
calmatives;
NO-synthase inhibitors;
agents for stimulating the synthesis of dermal or epidermal macromolecules and/or for preventing their degradation;
agents for stimulating fibroblast proliferation;
agents for stimulating keratinocyte proliferation;
muscle relaxants;
tensioning agents;
matting agents;
keratolytic agents;
desquamating agents;
moisturizers;
anti-inflammatory agents;
agents acting on the energy metabolism of cells;
insect repellants;
substance P or CGRP antagonists;
hair-loss counteractants and/or hair restorers;
anti-wrinkle agents.

Needless to say, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art. They may be in particular in the form of a simple or complex emulsion (O/W, W/O, O/W/O or W/O/W emulsion) such as a cream, a milk or a cream-gel. They may optionally be conditioned as an aerosol and may be in the form of a mousse or a spray.

The compositions according to the invention are preferably in the form of an oil-in-water or water-in-oil emulsion.

The emulsions generally contain at least one emulsifier chosen from amphoteric, anionic, cationic and nonionic emulsifiers, which are used alone or as a mixture. The emulsifiers are appropriately chosen according to the emulsion to be obtained (W/O or O/W). The emulsions may also contain stabilizers of other types, for instance fillers, gelling polymers or thickeners.

As emulsifying surfactants that may be used for the preparation of the W/O emulsions, examples that may be mentioned include sorbitan, glycerol or sugar alkyl esters or ethers; silicone surfactants, for instance dimethicone copolyols, such as the mixture of cyclomethicone and of dimethicone copolyol, sold under the name DC 5225 C by the company Dow Corning, and alkyldimethicone copolyols such as laurylmethicone copolyol sold under the name Dow Corning 5200 Formulation Aid by the company Dow Corning; cetyl-dimethicone copolyol, such as the product sold under the name Abil EM 90R by the company Goldschmidt, and the mixture of cetyldimethicone copolyol, of poly-glyceryl isostearate (4 mol) and of hexyl laurate, sold under the name Abil WE O9 by the company Goldschmidt.

One or more co-emulsifiers may also be added thereto, which may be chosen advantageously from the group comprising polyol alkyl esters.

Polyol alkyl esters that may especially be mentioned include polyethylene glycol esters, for instance PEG-30 dipolyhydroxystearate, such as the product sold under the name Arlacel P135 by the company ICI.

Glycerol and/or sorbitan esters that may especially be mentioned include, for example, polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters, for instance the mixture PEG-100 stearate/glyceryl stearate sold, for example, by the company ICI under the name Arlacel 165; oxyalkylenated (oxyethylenated and/or oxypropylenated) fatty alkyl ethers; sugar esters, for instance sucrose stearate; fatty alkyl ethers of sugars, especially polyalkylglucosides (APG) such as decylglucoside and laurylglucoside sold, for example, by the company Henkel under the respective names Plantaren 2000 and Plantaren 1200, cetostearyl glucoside optionally as a mixture with cetostearyl alcohol, sold, for example, under the name Montanov 68 by the company SEPPIC, under the name Tegocare CG90 by the company Goldschmidt and under the name Emulgade KE3302 by the company Henkel, and also arachidyl glucoside, for example in the form of a mixture of arachidyl alcohol, behenyl alcohol and arachidyl glucoside, sold under the name Montanov 202 by the company SEPPIC. According to one particular embodiment of the invention, the mixture of the alkylpolyglucoside as defined above with the corresponding fatty alcohol may be in the form of a self-emulsifying composition as described, for example, in document WO-A-92/06778.

Among the other emulsion stabilizers that will be used more particularly are isophthalic acid or sulfo-isophthalic acid polymers, and in particular phthalate/sulfoisophthalate/glycol copolymers, for example the diethylene glycol/phthalate/isophthalate/1,4-cyclo-hexanedimethanol copolymer (INCI name: Polyester-5) sold under the name Eastman AQ Polymer (AQ35S, AQ38S, AQ55S and AQ48 Ultra) by the company Eastman Chemical.

When it is an emulsion, the aqueous phase of this emulsion may comprise a nonionic vesicular dispersion prepared according to known processes (Bangham, Standish and Watkins, J. Mol. Biol. 13, 238 (1965), FR 2 315 991 and FR 2 416 008).

The compositions according to the invention find their application in a large number of treatments, especially cosmetic treatments, of the skin, the lips and the hair, including the scalp, especially for protecting and/or caring for the skin, the lips and/or the hair, and/or for making up the skin and/or the lips.

Another subject of the present invention consists of the use of the compositions according to the invention as defined above for the manufacture of cosmetic products for treating the skin, the lips, the nails, the hair, the eyelashes, the eyebrows and/or the scalp, especially care products, antisun products and makeup products.

The cosmetic compositions according to the invention may be used, for example, as makeup products.

The cosmetic compositions according to the invention may be used, for example, as care and/or antisun products for the face and/or the body, of liquid to semi-liquid consistency, such as milks, more or less rich creams, cream-gels and pastes. They may optionally be conditioned as an aerosol and may be in the form of a mousse or a spray.

The compositions according to the invention in the form of vaporizable fluid lotions in accordance with the invention are applied to the skin or the hair in the form of fine particles by means of pressurization devices. The devices in accordance with the invention are well known to those skilled in the art and comprise non-aerosol pumps or “atomizers”, aerosol containers comprising a propellant, and also aerosol pumps using compressed air as propellant. These containers are described in patents U.S. Pat. No. 4,077,441 and U.S. Pat. No. 4,850,517 (which form an integral part of the content of the description).

The compositions conditioned as aerosols in accordance with the invention generally contain conventional propellants, for instance the hydrofluoro compounds dichlorodifluoromethane and difluoroethane, dimethyl ether, isobutane, n-butane, propane or trichlorofluoromethane. They are preferably present in amounts ranging from 15% to 50% by weight relative to the total weight of the composition.

Concrete, but in no way limiting, examples illustrating the invention will now be given.

### EXAMPLE 1

### Preparation of 2,4,6-tris(methyltrimethyl-silyl 4′-aminobenzoate)-s-triazine

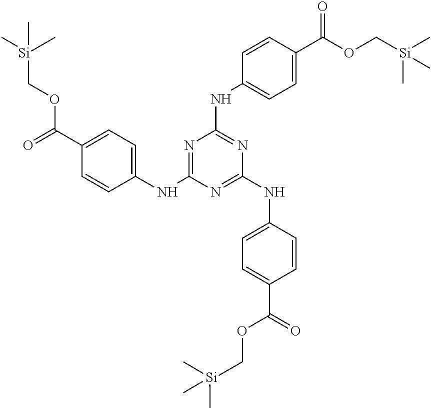

### First Step: Preparation of methyltrimethylsilyl para-aminobenzoate

Chloromethyltrimethylsilane (57.576 g, 0.471 mol) is added dropwise under nitrogen over 20 minutes to the potassium salt of para-aminobenzoic acid (75 g, 0.428 mol) dispersed in 500 ml of DMF in a reactor. The heterogeneous medium is refluxed for 4 hours. The DMF is evaporated off under vacuum. The residue is taken up in 300 ml of dichloromethane. The organic phase is washed three times with water. After adding heptane, an abundant precipitate forms. After filtering off and drying under vacuum, 149 g (78% yield) of methyltrimethylsilyl para-aminobenzoate are obtained in the form of a white solid, which is used in the following step without further purification.

### Second Step: Preparation of the Derivative of Example 1

While sparging with nitrogen at 0-5° C., the above derivative (3.08 g, 0.0138 mol) dissolved in 15 ml of toluene is introduced dropwise into a dispersion of cyanuric chloride (0.848 g, 4.59×10³ mol) in 15 ml of toluene. The mixture is then refluxed for 1 hour 30 minutes. After cooling, the white solid obtained is filtered off and recrystallized from an ethanol/water mixture. The derivative of Example 1 is thus obtained (2.39 g, 70% yield) in the form of a white powder:

UV (DMSO/ethanol) λₘₐₓ=314 nm; εₘₐₓ=126 660; E_{1%}=1700.

### EXAMPLE 2

### Preparation of 2,4-bis(methyltrimethylsilyl 4′-aminobenzoate)-6-(butyl 4″-aminobenzoate)-s-triazine

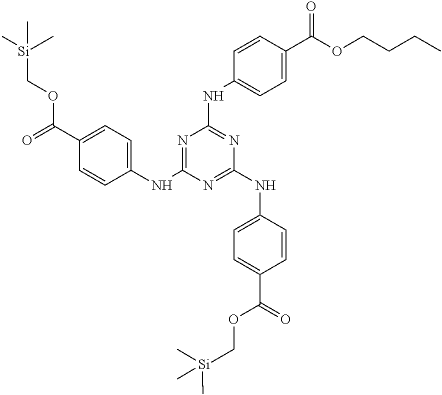

### First Step: Preparation of 2,4-dichloro-6-(butyl 4′-aminobenzoate)-s-triazine

To a suspension of cyanuric chloride (18.4 g, 0.1 mol) in 250 ml of acetone at a temperature of 5° C. are added alternately over 30 minutes, with mechanical stirring, a solution of butyl para-aminobenzoate (19.3 g, 0.1 mol) in 70 ml of acetone, and a solution of sodium bicarbonate (8.4 g, 0.1 mol) in 70 ml of water. The mixture is stirred at 5° C. for 30 minutes. The reaction mixture is filtered and the solid obtained is washed with water and dried. A white solid of 2,4-dichloro-6-(butyl 4′-aminobenzoate)-s-triazine is obtained (33.4 g, 98% yield, m.p. 248° C.) and is used in the following step without further purification.

### Second Step: Preparation of the Derivative of Example 2

While sparging with nitrogen, the heterogeneous medium of the above derivative (4.58 g, 0.0134 mol) and of methyltrimethylsilyl para-aminobenzoate (3 g, 0.268 mol) in 80 ml of toluene is refluxed for 5 hours minutes. After a phase of total dissolution, a precipitate forms. After cooling, the white solid obtained is filtered off and recrystallized from ethanol. The derivative of Example 2 is thus obtained (2.13 g, 22% yield) in the form of a white powder:

UV (DMSO/ethanol) λₘₐₓ=313 nm; εₘₐₓ=104 560; E_{1%}=1660.

### EXAMPLE 3

### Preparation of 2,4,6-tris(methyltrimethyl-silyl 4′-aminosalicylate)-s-triazine

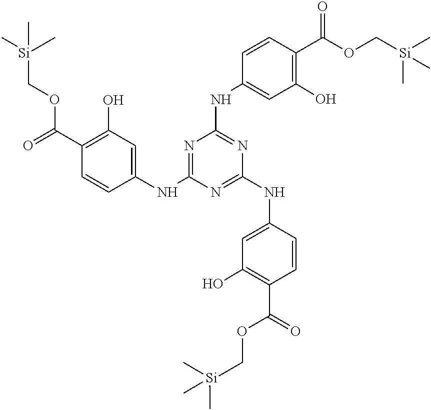

### First Step: Preparation of methyltrimethylsilyl 2-hydroxy-4-aminobenzoate

Chloromethyltrimethylsilane (12.2 g, 0.1 mol) is added dropwise over 20 minutes to a mixture of salicylic acid (15.3 g, 0.1 mol) and potassium carbonate (13.8 g, 0.1 mol) dispersed in 100 ml of DMF in a reactor. The heterogeneous medium is maintained at 95-100° C. for 2 hours. After cooling, the reaction mixture is diluted with 200 ml of water. It is extracted with dichloromethane. The organic phase is washed three times with water and dried over sodium sulfate. After evaporating off the solvent under vacuum, 16 g of an orange-coloured oil are obtained. This oil is purified by passing it through a bed of silica (eluent: 50/50 heptane/EtOAc). A beige-coloured oil is isolated, which crystallizes to give 10.4 g of methyltrimethylsilyl 2-hydroxy-4-aminobenzoate (43% yield), which is used in the following step without further purification.

### Second Step: Preparation of the Derivative of Example 3

While sparging with argon at 0-5° C., the above derivative (3.6 g, 0.015 mol) dissolved in 15 ml of toluene is introduced dropwise into a dispersion of cyanuric chloride (0.92 g, 5×10⁻³ mol) in 20 ml of toluene. The mixture is then refluxed for 3 hours. After cooling, the white solid obtained is filtered off and washed with ethyl acetate. The derivative of Example 3 is thus obtained (2.39 g, 88% yield) in the form of a white powder:

UV (DMSO/ethanol) λₘₐₓ=326 nm; εₘₐₓ=107 610; E_{1%}=1456,

UV (DMSO/ethanol) λₘₐₓ=300 nm; εₘₐₓ=74 400; E_{1%}=938.

### EXAMPLE 4

### Preparation of 2,4-bis(methyltrimethylsilyl 4′-aminobenzoate)-6-(para-methoxyphenyl)-s-triazine

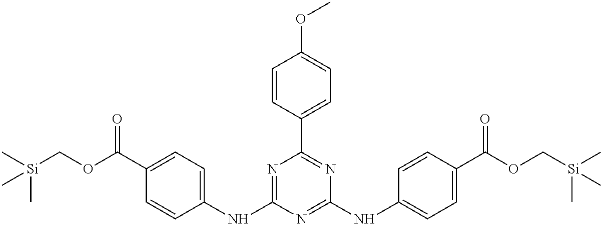

While sparging with nitrogen, a mixture of 2,4-dichloro-6-(4-methoxyphenyl)-1,3,5-triazine (1.72 g, 0.0067 mol) and of methyltrimethylsilyl para-aminobenzoate (3 g, 0.0134 mol) in 50 ml of toluene is introduced into a reactor. The mixture is gradually heated to reflux and is left at reflux for 1 hour 30 minutes. After cooling, the solid formed is filtered off and washed with hot ethanol. After drying, 3.58 g (85% yield) of the derivative of Example 5 are obtained in the form of a white powder:

UV (DMSO/ethanol) λₘₐₓ=303 nm; εₘₐₓ=82 700; E_{1%}=1313.

### EXAMPLE 5

### Preparation of 2,4-bis(methyltrimethylsilyl 4′-aminosalicylate)-6-(para-methoxyphenyl)-s-triazine

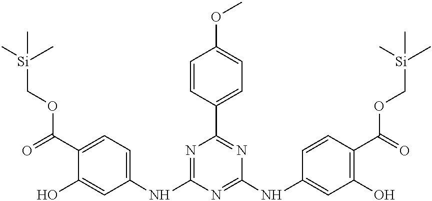

While sparging with nitrogen, a mixture of 2,4-dichloro-6-(4-methoxyphenyl)-1,3,5-triazine (1.3 g, 0.005 mol) and of methyltrimethylsilyl 2-hydroxy-4-aminobenzoate (2.4 g, 0.01 mol) in 10 ml of toluene is introduced into a reactor. The mixture is gradually heated to reflux and is left at reflux for 1 hour. After cooling, the solid formed is filtered off and washed with acetone. After drying, 3.2 g (97% yield) of the derivative of Example 6 are obtained in the form of a white powder:

UV (DMSO/ethanol) λₘₐₓ=329 nm; εₘₐₓ=61 370; E_{1%}=927,

UV (DMSO/ethanol) λₘₐₓ=316 nm; εₘₐₓ=70 570; E_{1%}=1066,

UV (DMSO/ethanol) λₘₐₓ=298 nm; εₘₐₓ=68 190; E_{1%}=1030.

### EXAMPLE 6

### Preparation of 2,4-bis(methyltrimethylsilyl 4′-aminobenzoate)-6-(4″-aminobenzylidenecamphor)-s-triazine

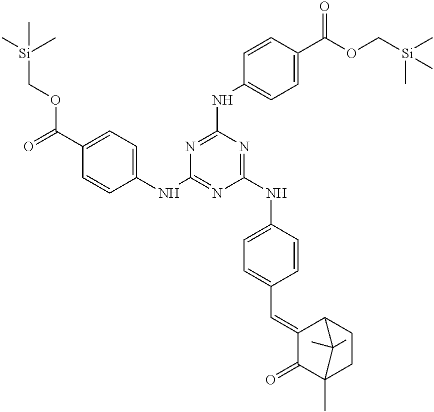

While sparging with nitrogen, a mixture of 2,4-dichloro-6-(4-methoxyphenyl)-1,3,5-triazine (2.7 g, 0.0067 mol) and of methyltrimethylsilyl para-aminobenzoate (3 g, 0.0134 mol) in 60 ml of toluene is introduced into a reactor. The mixture is gradually heated to reflux and is left at reflux for 2 hours. After cooling, the solid formed is filtered off and washed with hot heptane. After drying, 4.19 g (81% yield) of the derivative of Example 8 are obtained in the form of a white powder:

UV (DMSO/ethanol) λₘₐₓ=313 nm; εₘₐₓ=87 800; E_{1%}=1130,

UV (DMSO/ethanol) λₘₐₓ=345 nm; εₘₐₓ=41 500; E_{1%}=534.

### EXAMPLE 7

### Preparation of 2,4,6-tris(methyltrimethyl-silyl 4′-aminobenzamide)-s-triazine

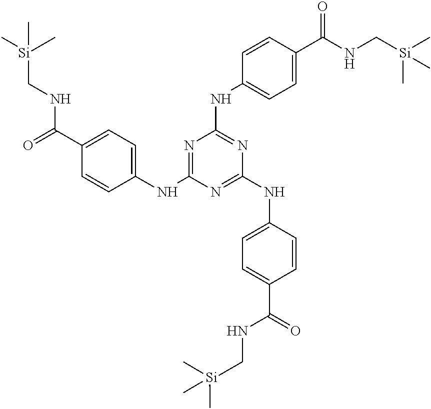

### First Step: Preparation of 4-nitro-N-(methyltrimethyl-silyl)benzamide

Methyltrimethylsilylamine (11 ml, 0.081 mol) is added dropwise over 20 minutes, under nitrogen, to a mixture of 4-nitrobenzoyl chloride (15 g, 0.081 mol) and triethylamine (11 ml, 0.081 mol) dissolved in 80 ml of dichloromethane in a reactor. The exothermic reaction is left at reflux for 2 hours. After cooling, the reaction mixture is poured into water. The resulting mixture is extracted with dichloromethane. The organic phase is washed three times with water and dried over sodium sulfate. After filtering and evaporating off the solvent under vacuum, 17.7 g of 4-nitro-N-(methyltrimethylsilyl)benzamide are obtained (87% yield) in the form of an orange-coloured powder, which is used in the following step without further purification.

### Second Step: Preparation of 4-amino-N-(methyltrimethyl-silyl)benzamide

Acetic acid (25 ml) is added dropwise, at a temperature below 40° C., to a mixture of the product of step 1 (9 g, 0.036 mol), acetic acid (18 ml), water (25 ml) and reduced iron (20 g, 0.36 mol) in a reactor. The exothermic reaction is left at 40° C. for 2 hours. After cooling, 100 ml of dichloromethane are added and the whole is filtered through Celite. The isolated organic phase is washed twice with water and dried over sodium sulfate. After filtering and evaporating off the solvent under vacuum, an orange-coloured powder is obtained, which is recrystallized from a 50/50 mixture of ethyl acetate and heptane. 6.16 g of 4-amino-N-(methyltrimethylsilyl)benzamide are obtained (77% yield; m.p. 126-127° C.) in the form of a light-beige powder, which is used in the following step without further purification.

### Third Step: Preparation of the Derivative of Example 7

While sparging with nitrogen at 0-5° C., the above derivative (4.1 g, 0.0184 mol) dissolved in 20 ml of toluene is introduced dropwise into a dispersion of cyanuric chloride (1.1 g, 6.1×10⁻³ mol) in 30 ml of toluene. The mixture is then refluxed for 3 hours. After cooling, the solid obtained is filtered off and washed with ethyl acetate. The derivative of Example 7 is thus obtained (3.26 g, 72% yield) in the form of a white powder.

UV (DMSO/ethanol) λₘₐₓ=303 nm; εₘₐₓ=83 330; E_{1%}=1123.

### EXAMPLE 8

### Comparative Tests of Dispersibility in an Oil of Two Insoluble Screening Agents 1 and 2

The dispersibility in an oil of the two insoluble organic screening agents below was studied.

### Compound 1 (Invention): 2,4,6-tris(methyltrimethylsilyl 4′-aminobenzoate)-s-triazine

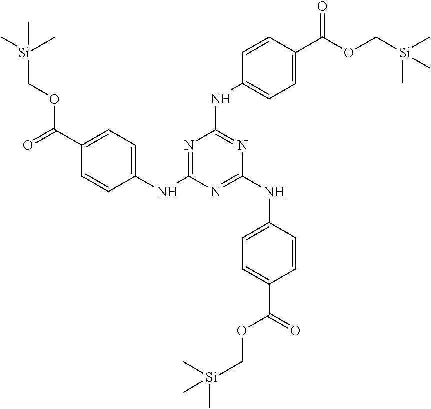

### Compound 2 (Outside the Invention): methylenebis(benzo-triazolyl)tetramethylbutylphenol, Sold in Solid Form Under the Trade Name Mixxim BB/100 by Fairmount Chemical, of Formula

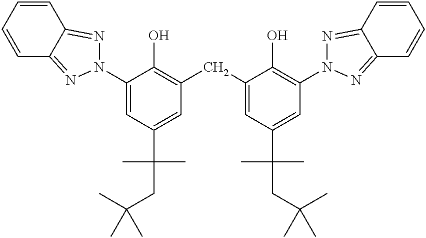

Two micronizations of these compounds 1 and 2 are performed by milling in oily medium (oil: phenylethyl benzoate—X-Tend 226 from ISP) by means of a mill without addition of dispersant, using a Dispermat ball mill.

### The Milling Conditions are as Follows:

Dispermat AE 06-C1 disperser-mill (Brant Industrie)
Dispersion: slurries containing 17% R056336A and 49% F17845 (difference in oil uptake) ˜3000 rpm ˜15 minutes
Milling under rapid conditions (30 minutes at 4500 rpm), not optimized (water-cooling circuit not operational) with 0.7-1.2 mm Zr₂O₂ balls.

### Results of Dispersibility of Compounds 1 and 2

1. Macroscopic Observation

Two oily dispersions in phenylethyl benzoate (X-Tend 226) comprising 1% by weight, respectively, of compound 1A or 2A are then prepared.

After three days at room temperature, it is observed that dispersion 1A according to the invention is homogeneous and shows no sign of sedimentation or of creaming, unlike dispersion 2A comprising Mixxim BB 100. After about one week, it is observed that, in dispersion 2A, compound 2 is totally insoluble in the phenylethyl benzoate, whereas dispersion 1A comprising compound 1 remains macroscopically stable at time to.

2. Microscopic Observation

Two oily dispersions 1A and 2B in phenylethyl benzoate comprising 10% by weight, respectively, of compound 1 or 2 are prepared.

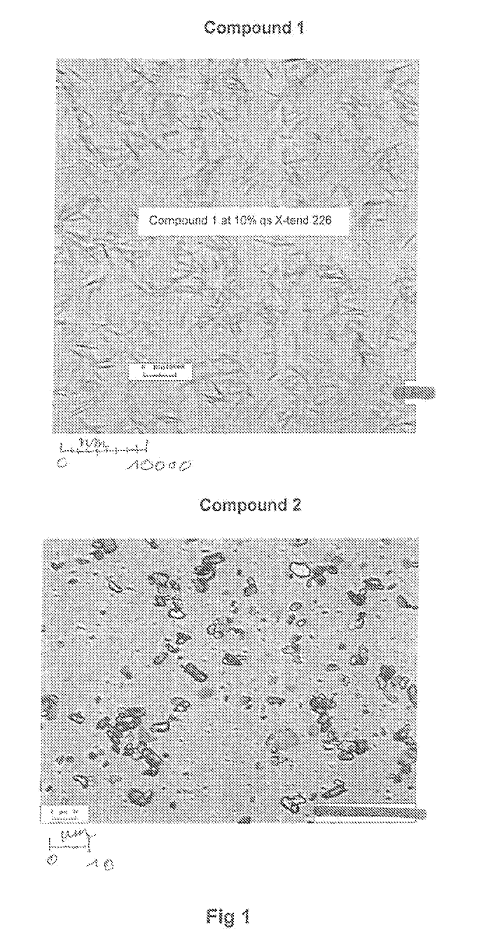

FIG. 1 shows that at time t0, dispersion 1B according to the invention comprising compound 1 is homogeneous and contains acicular (needle-shaped) particles, unlike the heterogeneous dispersion 2B comprising compound 2, which contains misshapen particles.

3. Spectral Measurements

The UV spectra in absorbance from 250 to 400 nm of dispersions 1B and 2B are acquired using a UV spectrometer equipped with an integration sphere, as a thin layer (10 μm optical path).

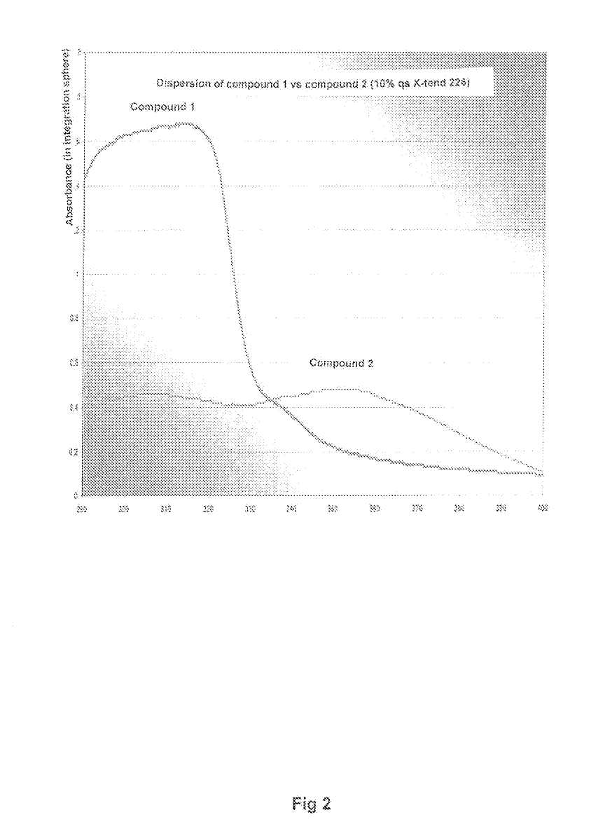

FIG. 2 shows markedly superior absorbance for dispersion 1B according to the invention comprising compound 2 compared with that for dispersion 2B comprising compound 2.

4. Granulometric Measurements

The granulometric results obtained by SLS using a Mastersizer granulometer reveal a very marked difference in particle size between compound 1 and compound 2, with particle sizes d(0.5), respectively, of 0.118 μm as opposed to 851 μm. This demonstrates that compound 1 is dispersible in the oil and may be micronized more readily without dispersant by means of grinding in an oily medium.

These results collectively demonstrate the markedly superior dispersibility of compound 1 according to the invention in the oil phenylethyl benzoate, when compared with that obtained with compound 2.

### EXAMPLE 9

### Antisun O/W emulsion

A concrete example of a cosmetic composition in the form of an emulsion of oil-in-water type is given below (the amounts are expressed as weight percentages relative to the total weight of the composition):

| | |
|---|---|
| Mixture of cetylstearyl alcohol and of | 7.0% |
| cetylstearyl alcohol oxyethylenated with | |
| 33 mol of ethylene oxide, sold under the | |
| trade name Sinnovax AO by Henkel | |
| Non-self-emulsifying glyceryl mono- and | 2.0% |
| distearate mixture | |
| Cetyl alcohol | 1.5% |
| Silicone oil | 1.5% |
| Diisopropyl adipate | 15% |
| Compound of Example 1 | 5.0% |
| Glycerol | 20.0% |
| Fragrance, preserving agents | qs |
| Water | qs 100% |

This composition was prepared in the following manner: after preparing the emulsion, the screening agent was dispersed at about 40° C. The cream obtained was then homogenized in a three-roll mill.

This composition absorbs in the UV-B range.

1. Compound corresponding to the general formula (I) below: 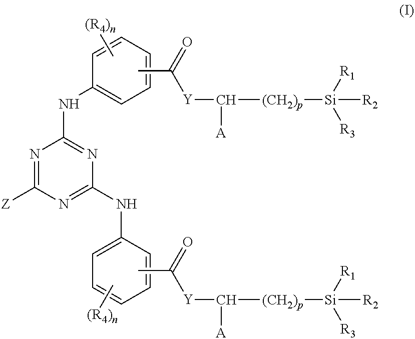
in which:
the radicals R₁, R₂ and R₃, which may be identical or different, represent a linear or branched, saturated or unsaturated C₁-C₁₂ alkyl radical, which may comprise one or more halogen atoms; a phenyl radical,
p is equal to 0 or 1,
Y denotes -0- or —NR₅,
the radical R₅ denotes hydrogen, a linear or branched 5 Ci-C₈ alkyl radical or a C₆-Ci₂ aryl radical,
A denotes hydrogen, a linear or branched, saturated or unsaturated C₁-C₈ alkyl radical, a phenyl radical or a group Si (CH₃)₃, with the proviso that when A is Si(CH₃)₃, then p=0 and R₁, R₂ and R₃ are methyl,
the radical R₄ denotes a linear or branched, saturated or unsaturated C₁-C₂₀ alkyl radical, an OH radical, a linear or branched C₁-C₂₀ alkoxy radical or a group —(C═O)—Y—R₆,
n is equal to 0, 1 or 2,
the radical R₆ denotes hydrogen or a linear or branched C₁-C₂₀ alkyl radical,
the group (CO)—Y—CHA-(CH₂)ₚ—SiRiR₂R₃ may be in the meta or para position relative to the amino group,
Z represents:
(i) a linear or branched C₁-C₂₀ alkyl group,
(ii) a linear or branched C₂-C₂₀ hydroxyalkyl group,
(iii) a linear or branched C₁-C₂₀ alkoxy group,
(iv) a para-alkoxyphenyl group of the type: 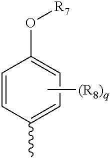
in which R₇ represents
hydrogen;
a linear or branched C₁-C₂₀ alkyl group;
a C₆-C₁₀ aryl group;
a group —(CH₂CHR₉—O)ₘR₁₀ or a group —CH₂—CH(OH)—CH₂—O—R₁₀ in which:
R₉ represents hydrogen or methyl,
R₁₀ represents hydrogen or a linear or branched C₁-C₈ alkyl group, m=1-20,
the radicals R₈, which may be identical or different, represent a linear or branched C₁-C₈ alkyl group, an OH radical or a linear or branched C₁-C₈ alkoxy group, this alkoxy group possibly forming with the ortho group OR₇ a C₁-C₂ dioxyalkylene ring, with the following exclusion that R₈ cannot be a hydroxyl radical in the meta position relative to the group OR₇,
q=0-3,
(v) the radical NHR₁₁ or the radical N(R₁₁)₂ with R₁₁ being a linear or branched C₁-C₂₀ alkyl radical,
vi) the group: 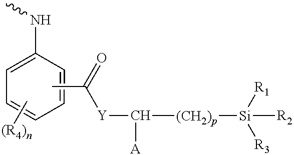
(vii) or the radical —NH—W in which W represents a chromophore for screening out UV radiation, chosen in particular from the group constituted by a benzylidenecamphor, a benzalmalonate, a benzoate, a salicylate, an anthranilate, a benzotriazole, a benzimidazole, a benzoxazole, a benzothiazole, a cinnamate or a cinnamonitrile.

2. Compound according to claim 1, having at least one of the following characteristics:
R₁ to R₃ denote a C₁-C₄ alkyl,
p is 0,
A is H or Si(CH₃)₃,
Y is —O— or —NH—,
n is 0 or 1,
Z is a silane aminobenzoate or para-methoxyphenyl group,
F is a benzoate or benzylidenecamphor group.

3. Compound according to claim 2, chosen from the compounds of formulae (a) to (g) below:

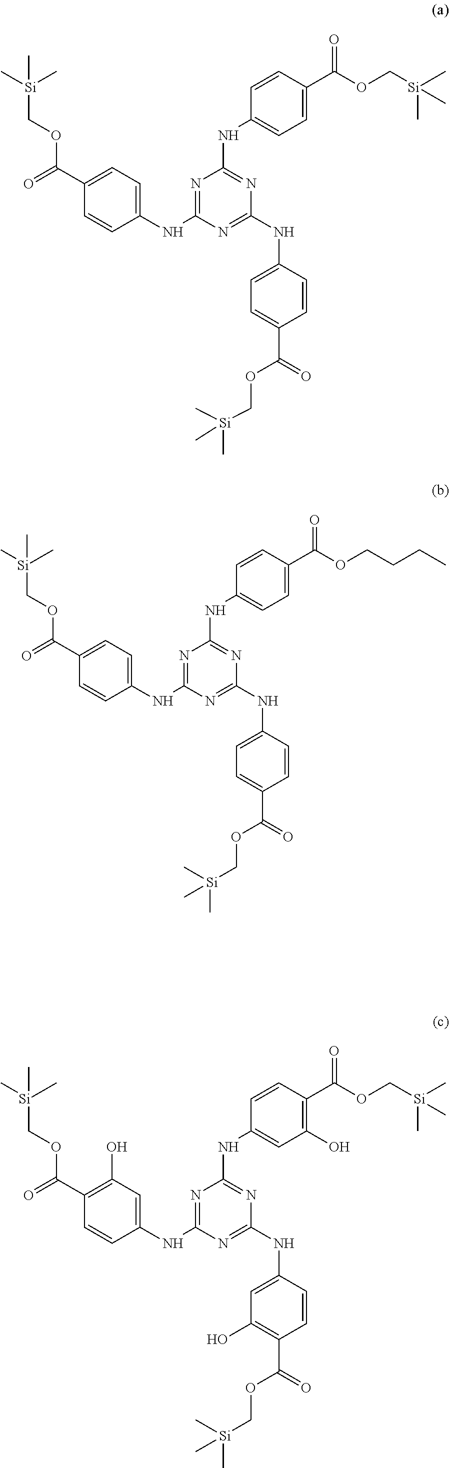

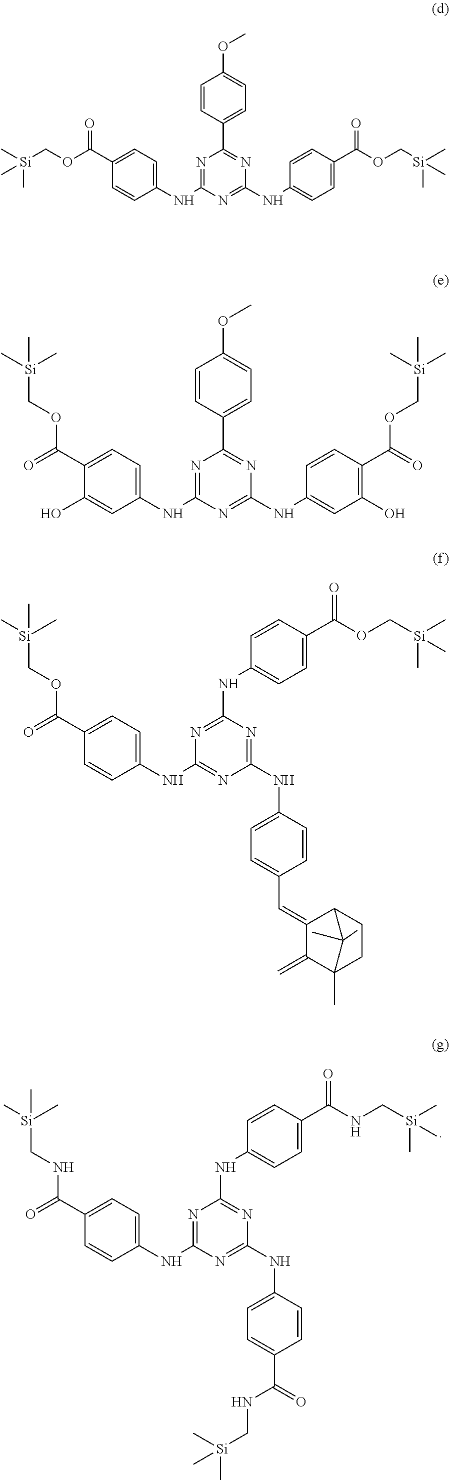

4. Compound according to claim 1, characterized in that it is in micronized form and that the mean particle size is between 10 nm and 5 μm.

5. Compound according to claim 4, in which the particles have a mean size of between 10 nm and 2 μm and more preferentially between 20 nm and 2 μm.

6. Cosmetic or dermatological composition for photoprotecting keratin materials, containing, in a cosmetically acceptable medium, at least one compound of formula (I) as defined in claim 1.

7. Composition according to claim 6, in which the compound (s) of formula (I) is (are) present in concentrations ranging from 0.01% to 20% by weight relative to the total weight of the composition.

8. Composition according to claim 6, also comprising at least one additional UV-A-active and/or UV-B-active organic or mineral UV-screening agent, which is water-soluble or liposoluble, or insoluble in the commonly used cosmetic solvents.

9. Composition according to claim 8, in which the said additional organic screening agent (s) is (are) chosen from:
Butylmethoxydibenzoylmethane,
Ethylhexyl methoxycinnamate,
Homosalate,
Ethylhexyl salicylate,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid.
Disodium phenyldibenzimidazoletetrasulfonate,
Methylenebis(benzotriazolyl)tetramethylbutylphenol,
Ethylhexyl triazone,
bis-Ethylhexyloxyphenol methoxyphenyl triazine,
Diethylhexyl butamido triazone,
2,4,6-tris(Dineopentyl 4′-aminobenzalmalonate)-s-triazine,
2,4,6-tris(Diisobutyl 4′-aminobenzalmalonate)-s-triazine,
2,4,6-tris(Biphenyl-4-yl-1,3,5-triazine),
2,4,6-tris(Terphenyl)-1,3,5-triazine
Drometrizole trisiloxane.
Polysilicone-15,
Dineopentyl 4′-methoxybenzalmalonate,
1,1-Dicarboxy(2,2′-dimethylpropyl)-4,4-diphenylbutadiene.
2,4-Bis[5-1 (dimethylpropyl)benzoxazol-2-yl (4-phenyl)imino]-6-(2-ethylhexyl)imino-1, 3.
5-triazine,
and mixtures thereof.

10. Composition according to claim 8, in which the mineral screening agents, are chosen from coated or uncoated metal oxide pigments in which the mean size of the primary particles ranges between 5 nm and 100 nm.

11. Composition according to claim 10, in which the mineral screening agents are chosen from coated or uncoated titanium oxide, iron oxide, zinc oxide, zirconium oxide or cerium oxide pigments.

12. Composition according to claim 6, characterized in that it also contains at least one self-tanning agent.

13. Composition according to claim 6, characterized in that it contains at least one fatty phase.

14. Composition according to claim 13, characterized in that it is in the form of an oil-in-water or water-in-oil emulsion.

15. Cosmetic use of a compound of formula (I) according to claim 1, as an agent for screening out UV-B radiation.

16. Compound according to claim 2, characterized in that it is in micronized form and that the mean particle size is between 10 nm and 5 μm.

17. Compound according to claim 3, characterized in that it is in micronized form and that the mean particle size is between 10 nm and 5 μm.

18. Cosmetic or dermatological composition for photoprotecting keratin materials, containing, in a cosmetically acceptable medium, at least one compound of formula (I) as defined in claim 2.

19. Cosmetic or dermatological composition for photoprotecting keratin materials, containing, in a cosmetically acceptable medium, at least one compound of formula (I) as defined in claim 2.

20. Composition according to claim 7, also comprising at least one additional UV-A-active and/or UV-B-active organic or mineral UV-screening agent, which is water-soluble or liposoluble, or insoluble in the commonly used cosmetic solvents.
